(19) United States
(12) Patent Application Publication
(10) Pub. No.: US 2014/0127299 A1
(43) Pub. Date: May 8, 2014

(54) GRAS Enteric Coating Formulations and Methods of Making and Using Same

(71) Applicant: NaZura BioHealth, Inc., San Diego, CA (US)

(72) Inventor: Stephen Kwaku Dordunoo, Baltimore, MD (US)

(21) Appl. No.: 14/074,646

(22) Filed: Nov. 7, 2013

Related U.S. Application Data
(60) Provisional application No. 61/723,737, filed on Nov. 7, 2012.
(60) Provisional application No. 61/738,947, filed on Dec. 18, 2012.

(51) Int. Cl. A61K 9/28

(52) U.S. Cl. CPC A61K 9/282; A61K 9/2866; A61K 9/2893
USPC 424/480; 514/781; 514/784; 424/475; 427/2.21

(57) ABSTRACT

Disclosed herein are food-grade enteric coating compositions designed to release pharmaceutical and/or nutraceutical products at various regions of the intestines, wherein said compositions comprise a film former and a pore former. Also disclosed herein are methods of making and using same.

### FIELD OF INVENTION

The present invention relates to food-grade formulations suitable for use as an enteric coating for the release of an active agent from a core solid dosage form at various regions in the intestines, e.g., at a pH between 5.5 and 6.5, and methods of making and using same. More particularly, the present invention relates to formulations (and methods of making and using same) wherein the formulation comprises Generally Recognized as Safe (GRAS) materials.

### BACKGROUND OF THE INVENTION

Enteric film coatings are applied to oral dosage forms to delay the release of active ingredients until the dosage form has passed beyond the acidic environment of the stomach or other intestinal pHs. The chemical environment of the stomach and gastric physiology are highly variable, subject to multiple factors such as disease state, medication, age, and prandial state. For example in the fasted state stomach, the pH may be less than 2 in healthy individuals, and gastric emptying of oral dosage forms generally occurs within 30 to 60 minutes. However in a postprandial state, gastric emptying of enteric coated oral dosage forms may be delayed for 2 to 4 hours and gastric pH may rise above 2 (e.g. a pH 4) for variable amounts of time.

There is a long history of use of enteric coatings on tablets and smaller multiparticulate dosage forms in the pharmaceutical industry. Examples of enteric film coatings include methacrylic acid copolymers, polyvinyl acetate phthalate, cellulose acetate phthalate, hydroxypropyl ethylcellulose phthalate and hydroxypropyl ethylcellulose acetylsuccinate. However, none of the above polymers are found in the Food Chemicals Codex (FCC) and none of the above polymers have direct food additive status or have Generally Regarded as Safe (GRAS) status. Accordingly, food-grade enteric coatings that release a therapeutic agent at targeted regions of the gastrointestinal track are still needed.

Several strategies have been developed to provide for food grade enteric coatings for nutraceuticals and other items classified as food. One example is shellac. Shellac is a natural, food approved, resinous material obtained from the exudate of the insect Laccifer lacca. It is a complex mixture of materials. The two main components with enteric properties being shelloic and aleuritic acid. Shellac is insoluble in water but soluble in organic solvents including ethanol. As shellac is insoluble in acidic conditions but soluble at higher pH levels (i.e., pH greater than about 6.5) it would appear to be suitable as an enteric coating material. In practice, however, delayed disintegration and delayed drug release occurs in vivo as the shellac coat is typically not soluble in the upper intestine. Shellac commonly does not behave in a typical enteric coating manner and instead behaves more like an erodible coating, dissolving as a function of time rather than of pH.

An alternative approach is the use an aqueous ethylcellulose (EC) based pseudo-latex in conjunction with sodium alginate. This product is marketed as Nutrateric™ nutritional enteric coating system by Colorcon Inc. of Westpoint, Pa. However, these systems do not address directly the need for an enteric food grade coating that can target release of an active agent at various points beyond the stomach.

Accordingly, the need still exists in the art for a pH sensitive, food grade enteric coating formulation designed to release pharmaceutical and/or nutraceutical products at targeted locations in the intestines.

### SUMMARY OF INVENTION

The field of the invention generally relates to food-grade enteric coating compositions designed to release an active agent, e.g., a pharmaceutical agent, a nutraceutical, a dietary supplement, a nutritional supplement, a medical food, etc., contained in a core solid dosage form at various regions of the intestines. The enteric coating formulations disclosed herein comprise at least one film former and one or more pore formers. Preferably, the subject enteric coating coatings release an active agent from a core solid dosage form at a pH between about 4.0 and about 8.0, more preferably at a pH between about 5.0 and 7.0, still more preferably at a pH between about 5.5 and 6.5, as demonstrated herein.

The enteric coating compositions of the present invention preferably comprise from about 20% to about 90% (wt/wt) and more preferably from about 30% to about 60% (wt/wt) of a film former. In another embodiment, the film former provides about 25%, 30%, 35%, 40%, 45%, 50%, or 55% (wt/wt) of the total solids in the enteric coating composition. In one embodiment, the film former is a pH dependent polymer. In another embodiment, the film former is a pH-independent polymer. In preferred embodiments, the film former is a GRAS compound. In an exemplary embodiment, the film former is selected from the group consisting of shellac and ethyl cellulose. In a particularly preferred embodiment, the film former is shellac.

The enteric coating compositions as disclosed herein further comprise a pore former; preferably from about 5% (wt/wt) to about 80% (wt/wt), and more preferably from about 10% (wt/wt) to about 60% (wt/wt) of the pore former. In another embodiment, the pore former provides about 10% (wt/wt) to about 60% (wt/wt), and more preferably about 10% (wt/wt) to about 20%, 30% or 40% (wt/wt) of the total solids in the enteric coating composition. In some embodiments, the pore former has very low solubility at low pH. In preferred embodiments, the pore former has a pKa greater than about 3.0, more preferably greater than about 3.5, and still more preferably greater than about 4.0 or 4.5. In some embodiments the pore former has a pKa between about 3 and 6, and more preferably between about 4 and 5. In some embodiments, the pore former has a pKa less than about 6. In one embodiment, the pore former is an organic acid, salt, or derivative thereof, preferably an organic acid, salt, or derivative thereof that is Generally Recognized as Safe (GRAS). In a preferred embodiment, the pore former is an organic acid selected from the group consisting of sorbic acid, benzoic acid and succinic acid. In a particularly preferred embodiment, the organic acid is sorbic acid.

The enteric coating compositions as described herein may advantageously further comprise a plasticizer and/or an anti-caking agent. For example, plasticizers such as triethyl citrate or polyethylene glycol as well as anti-caking agents such as glyceryl monostearate or talc may be included in the coating composition. In one embodiment, the enteric coating composition disclosed herein comprises up to about 20% (wt/wt) of either or both the plasticizer and the anti-caking agent. In another embodiment, the enteric coating comprises up to about 10% (wt/wt) plasticizer and/or about 10% (wt/wt) anti-caking agent.

The enteric coating compositions as described herein may be in the form of a spray solution, a suspension, or a solid enteric coating surrounding a core solid dosage form containing the active agent. In one embodiment, the enteric coating composition is in the form of a spray solution, preferably with a pH between about 7 and about 8. In another embodiment, the enteric coating composition is in the form of a suspension, preferably with a pH between about 2 and about 4. In a further embodiment, the enteric coating composition is in solid form. Preferably, the solid enteric coating composition is stable, e.g., releases less than about 10% of the active agent at a pH less than 2 for at least 1 hour.

Also provided are methods of preparing delayed-release solid dosage forms, comprising applying the enteric coating compositions disclosed herein to a core solid dosage form comprising the active agent. In a preferred embodiment, the enteric coating composition is applied to achieve about a 2% to about a 50% (wt/wt) target weight gain (TWG) to the desired dosage form. For core tablets and capsules, the enteric coating can be applied to achieve about a 1.5% to about a 5% or 6% (wt/wt) TWG, more preferably about a 2% to about a 4% TWG. For granules and other multi-particulate dosage forms up to 20 or 30% (wt/wt) TWG or more can be applied, preferably from 20%-50% (wt/wt) TWG, more preferably from 30%-50% (wt/wt) TWG. In another embodiment, the method further comprises applying a top coat, which may be another film former, to the enteric coating composition. In one embodiment, the top coat is a GRAS film forming ingredient. In another embodiment, the top coat is selected from the group consisting of hydroxypropylmethyl-cellulose (HMPC), polyvinyl alcohol (PVA) or Eudragit E.

Also disclosed herein are delayed-release solid dosage forms comprising (a) a core solid dosage form comprising at least one active agent, e.g., a pharmaceutical agent, a nutraceutical, a dietary supplement, a nutritional supplement, or a medical food, and (b) an enteric coating comprising at least one film former and at least one pore former as described herein, wherein the pore former comprises an organic acid having a pKa greater than about 3.0, more preferably greater than about 3.5, and still more preferably greater than about 4.0 or 4.5. In one embodiment, the core solid dosage form is selected from the group consisting of a capsule, tablet, mini-tablet, soft gel or granule. In preferred embodiments, the capsule, tablet, or soft gel comprises about 2% (wt/wt) to about 4% (wt/wt) TWG enteric coating.

Also provided are methods for delivering active agents to the intestine of a subject in need thereof, comprising administering to the subject a delayed-release solid dosage form comprising a core solid dosage form containing at least one active agent, wherein the core solid dosage form is surrounded by an enteric coating comprising at least one film former and at least one pore former as described herein. In preferred embodiments, the pore former comprises an organic acid having a pKa greater than about 3.0, more preferably greater than about 3.5, and still more preferably greater than about 4.0 or 4.5.

### BRIEF DESCRIPTION OF THE DRAWINGS

FIG. 1: Graph Showing Metformin Release below 5% after 2 hours when pH is less than or equal to 6.0 but is 15-45% after 2 Hr when pH is 6.5 or Greater. Shown in FIG. 1 is the amount of metformin released (percent; y-axis) over time (hours; x-axis) by core solid dosage forms coated with an enteric coating composition comprising shellac 60% and potassium sorbate 40% at a 2.4% target weight gain, and which are placed at a pH of 5 (♦), 5.5 (▪), 6 (▴), 6.5 ( [symbol] ), 6.8 ( [symbol] ) or 7 ().

FIG. 2: Graph Showing Metformin Release at pH 6.8—Lowest at 3% Coating (17%@2 Hr) and Highest at 2.2% Coating (35%@2 Hr). Shown in FIG. 2 is the amount of metformin released (percent; y-axis) over time (hours; x-axis) by core solid dosage forms coated with an enteric coating composition comprising shellac 60%:potassium sorbate 40% at a 2.2% (♦), 2.4% (▪), 2.6% (568), 2.8 ( [symbol] ) or 3.0 ( [symbol] ) weight gain, and which are placed at a pH of 6.8.

FIG. 3. Graph of Metformin Dissolution Profile at a pH of 6.8. Shown in FIG. 3 is the amount of metformin released (percent; y-axis) over time (hours; x-axis) by core solid dosage forms coated with an enteric coating composition comprising 40% shellac:60% potassium sorbate at a 2.4% (▪), 2.6% (▴), or 2.8 ( [symbol] ) weight gain, and which are placed at a pH of 6.8 after acid exposure for 2 hrs.

FIG. 4. Effect of coating level on dissolution of metformin from shellac 60%-benzoate 40% enterically-coated core tablets (in acid for 2 hours followed by buffer at pH6) showing faster release at lower coating levels compared with higher coating levels Shown in FIG. 4 is the amount of metformin released (Amount released (%); y-axis) over time (hours; x-axis) by core tablets that are coated with an enteric coating composition comprising 40% shellac:benzoic acid at a 2.0 (♦), 2.2% (▪), 2.4% (▴), 2.6 ( [symbol] ), 2.8 ( [symbol] ) or 2.9% (•) weight gain, and which are placed at a pH of 6.

FIG. 5. Comparison of metformin release from Shellac 60%-potassium sorbate 40% in acid for 2 hours followed by buffer pH 6.8 and Shellac 60%-sodium benzoate 40% in acid for 2 hours followed by buffer at pH 6 showing faster release from the benzoate-based coated tablets at pH 6 compared with the sorbate-based coated tablets at pH 6.8 Shown in FIG. 5 is the amount of metformin released (Amount released (%); y-axis) over time (hours; x-axis) by core tablets that are either (1) coated with an enteric coating composition comprising shellac 60%:benzoate 40% acid at a 2.4% weight gain and which are placed at a pH of 6 ( [symbol] ) or (2) coated with an enteric coating composition comprising shellac 60%:sorbate 40% at a 2.4% target weight gain and which are placed at a pH of 6.8 (▴).

FIG. 6. shows the release of IMP from mini-tablets coated with 20% sorbate in shellac coating solution to 5% target weight gain. This batch of mini-tablets resisted drug release in 0.1M HCl for 2 hours with less than 10% release at an intermediate pH of 3 and the rest of the drug released at pH 5.5 over 30-60 minutes.

FIG. 7. shows drug release from mini-tablets coated with 20% sorbate in shellac solution at 7.5% TWG. This batch of tablets resisted drug release in 0.1M HCl (pH 1.2) for 2 hours and at pH 5.5 for 1 hour but released the drug at pH 6.5

### DETAILED DESCRIPTION

The present invention relates to targeting active agents to various regions of the gastrointestinal tract with delayed-release solid dosage forms comprising an enteric coating as described herein. In particular, provided herein are food-grade enteric coating compositions designed to release pharmaceutical and/or nutraceutical products at various regions of the intestines. The enteric coating formulation includes at least one film former such as shellac or ethylcellulose, and one or more pore formers with low solubility at acidic pH and pKa values between about 3 and about 6, and more preferably between about 4 and 5. Optionally, plasticizers such as triethyl citrate or polyethylene glycol as well as anti-caking agents such are glyceryl monostearate or talc may also be included in the enteric coating compositions.

In preferred embodiments, the film former is present in a range of approximately 30% to 90% w/w and may itself be a pH-dependent or a pH-independent polymer. The pore former is preferably a food-grade substance, e.g., sorbic acid, benzoic acid, succinic acid, salts thereof, and derivatives thereof, and is generally present in a range of approximately 15% to 80% (w/w). When applied to orally ingestible substrates such as pharmaceutical tablets and dietary supplements, the composition forms an enteric coating. The enteric coating composition may be applied onto any oral solid dosage forms, such as pharmaceutical tablets and dietary supplements, to provide a delayed release enteric film. The enteric coating composition may be applied to core solid dosage forms including tablets, capsules, soft gels, or granules at coating levels of from 1.8 to 50% target weight gain depending on the type of solid dosage form.

Film Formers

Suitable film formers for use in the subject compositions and methods include, e.g., pH-dependent polymers, water-insoluble polymers, and low-melting hydrophobic materials, copolymers thereof, and mixtures thereof. In preferred embodiments, the film former is a compound that is Generally Recognized as Safe (“GRAS”) by the U.S. Food and Drug Administration, e.g., can be found within the FDA's database of GRAS substances (SCOGS), including, e.g., shellac and ethyl cellulose.

Examples of suitable water-insoluble polymers include, but are not limited to, ethyl cellulose, polyvinyl alcohols, polyvinyl acetate, polycaprolactones, cellulose acetate and its derivatives, acrylates, methacrylates, acrylic acid copolymers, copolymers thereof, and mixtures thereof. Suitable low-melting hydrophobic materials include, but are not limited to, fats, fatty acid esters, phospholipids, waxes, and mixtures thereof. Examples of suitable fats include, but are not limited to, hydrogenated vegetable oils such as for example cocoa butter, hydrogenated palm kernel oil, hydrogenated cottonseed oil, hydrogenated sunflower oil, and hydrogenated soybean oil, free fatty acids and their salts, and mixtures thereof. Examples of suitable fatty acid esters include, but are not limited to, sucrose fatty acid esters, mono-, di-, and tri-glycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, stearoyl macrogol-32 glycerides, and mixtures thereof. Examples of suitable phospholipids include phosphotidyl choline, phosphotidyl serene, phosphotidyl enositol, phosphotidic acid, and mixtures thereof. Examples of suitable waxes include, but are not limited to, carnauba wax, spermaceti wax, beeswax, candelilla wax, shellac wax, microcrystalline wax, and paraffin wax; fat-containing mixtures such as chocolate, and mixtures thereof. In one preferred embodiment, the film former is ethyl cellulose.

Suitable pH-dependent polymers for use as film formers include, but are not limited to, enteric cellulose derivatives such as hydroxypropyl methylcellulose phthalate, hydroxypropyl methylcellulose acetate succinate, cellulose acetate phthalate; natural resins such as shellac and zein; enteric acetate derivatives such as polyvinylacetate phthalate, cellulose acetate phthalate, acetaldehyde dimethylcellulose acetate; and enteric acrylate derivatives such as polymethacrylate-based polymers such as poly(methacrylic acid, methyl methacrylate) 1:2 (which is commercially under the tradename EUDRAGIT™), and poly(methacrylic acid, methyl methacrylate) 1:1 (which is commercially available under the tradename EUDRAGIT™), and mixtures thereof.

In one preferred embodiment, the enteric coating composition comprises shellac as a film former. Examples of shellacs include, but are not limited to, dewaxed bleached shellacs, dewaxed and decolorized shellacs (dewaxed orange shellac), and all USP shellacs. The shellac may be in an aqueous salt form or free acid form. For instance, the shellac may be an aqueous alkali salt of shellac. The shellac may also be an aqueous ammonium salt of shellac. The shellac may be formed out of water and not alcohol. The aqueous shellac may have at least about 5%, at least about 10%, at least about 15%, and at least about 20% solids. The aqueous shellac may have less than about 30% or less than about 25% solids. The aqueous shellac may be in solution at a range of about 5% to about 30% solids. The aqueous shellac may have about 20% to about 30% solids, particularly about 25% solids. Examples of commercially-available shellacs include, but are not limited to, MarCoat™ 125 (available from Emerson Resources) and Aqueous 125 (available from Parker Ingredients). MarCoat™ 125 contains dewaxed and decolorized shellac, methyl paraben, propyl paraben (as preservatives), isopropyl alcohol, and water. Aqueous 125 contains ammoniated shellac, denatured alcohol, potassium sorbate (an anti-oxidant or preservative), and water. Ammoniated shellac may be used in one embodiment. Commonly, an ammoniated shellac (as approved for food use) may exhibit different solubility characteristics in various pHs, may be readily available and may be economical.

Dewaxed orange shellac and refined bleached shellac may be used because of their low wax content. Orange shellac commonly has an acid value of about 68-71 and tends to form a better, glossier, and more impervious film. Bleached shellac commonly has an acid value of about 78-90 and tends to get tacky and forms a more permeable film. Both grades may be prepared as aqueous solutions using, for example, ammonium carbonate and/or ammonium hydroxide to solubilize them in water, and, if necessary a mixture of alcohol and water. Such aqueous solutions may comprise at least about 5%, at least about 10%, at least about 15%, or at least about 20% solids. The aqueous solutions may comprise less than about 30%, less than about 25%, less than about 24%, less than about 23%, less than about 22%, less than about 21%, less than about 20%, less than about 15%, or less than about 10% solids. This includes about 5% to about 30%, about 5% to about 25%, and about 10% to about 25% solids. The aqueous solutions may be prepared at temperatures of at least about 15° C., at least about 20° C., at least about 25° C., at least about 30° C., at least about 40° C., at least about 50° C., or at least about 60° C. The aqueous solutions may be prepared at temperatures of less than about 85° C., less than about 80° C., less than about 75° C., less than about 70° C., less than about 65° C., less than about 60° C., or less than about 50° C. This includes temperatures of about 15° C. to about 85° C., and about 20° C. to about 80° C.

In alternative embodiments, the enteric coating composition comprises a water-insoluble copolymer which is a copolymer composed of free-radical polymerized units of more than 95% by weight, in particular to an extent of at least 98% by weight, preferably to an extent of at least 99% by weight, in particular to an extent of at least 99% by weight, more preferably to an extent of 100% by weight, of (meth)acrylate monomers with neutral radicals, especially C₁₋₄-alkyl radicals. These kinds of polymers do not dissolve in water or are only swellable in water over the whole range of pH 1-14.

Suitable (meth)acrylate monomers with neutral radicals are, for example, methyl methacrylate, ethyl meth acrylate, butyl methacrylate, methyl acrylate, ethyl acrylate, butyl acrylate. Preference is given to methyl methacrylate, ethyl acrylate and methyl acrylate. Methacrylate monomers with anionic radicals, for example acrylic acid and/or methacrylic acid, may be present in small amounts of less than 5% by weight, preferably not more than 2% by weight, more preferably not more than 1 or 0.05 to 1% by weight.

In a further embodiment, one or more film formers can also be applied as part of an additional topcoat to a core solid dosage form already coated with an enteric coating composition as described herein. In one embodiment, the top coat is a Generally Recognized as Safe (GRAS) film former. In another embodiment, the top coat is selected from the group consisting of hydroxypropylmethyl-cellulose (HMPC), polyvinyl alcohol (PVA) or Eudragit E.

Pore Formers

A pore former as disclosed herein may generally be an organic acid, preferably a GRAS organic acid. In preferred embodiments, pore formers are solid organic acids having very low solubility at acidic conditions (e.g., at a pH of about 1 to about 3) and having a pKa greater than about 3.0, preferably greater than about 3.5, and more preferably greater than about 4.0 or 4.5. In some embodiments, the pore former has a pKa value between about 3 and 6, and more preferably between about 4 and 5.

Non-limiting examples of organic acids for use in the subject invention include sorbic acid, benzoic acid, succinic acid, salts thereof, and derivatives thereof, such as, e.g. their sodium, potassium, ammonium, lithium, or calcium salts. The type and amount of acid and/or salt thereof controls in part the pH release characteristics of the active agent from the solid dosage form, among other considerations. In general, organic acids having a higher pKa will produce an enteric coating releasing at a higher pH range, all other factors being equal. Preferred organic acids for use in the subject invention provide for delayed release of active agents from core solid dosage forms at a pH between about 4.0 and 8.0, more preferably between about 5.0 and 7.0, still more preferably between about 5.5 and 6.5, as demonstrated herein.

Plasticizers

Examples of plasticizers include, but are not limited to, fatty acids, water-soluble plasticizers, water-insoluble plasticizers, triethyl citrate, triacetin, glycerin, propylene glycol, polypropylene glycol, polyethylene glycol (molecular weights of about 300 to about 8000), dibutyl sebacate, triglycerides, medium chain triglycerides (e.g., fractionated coconut oil), acetylated monoglycerides, glycerol monostearates, glycerin monostearate, oleic acid, polysorbates (such as polysorbate 80), stearic acid, sorbitol, tributyl citrate, acetyltributyl citrate, dibutyl phthalate, triethyl citrate, triethanolamine, and combinations thereof. A plasticizer may modify the flexibility of the film formed to suit dosage requirements. A plasticizer may enhance the film characteristics of the enteric coating, such as adhesion, flexibility, permeability, etc.

Anti-Caking Agents

Examples of anti-caking agents include, but are not limited to, aluminum hydrate, acetylated glycerides, diglycerides, acetylated monoglyceride, polyvinylpyrrolidone, sorbitan monostearate, polyglycerol esters, ethyl acetate, glyceryl monostearate, monoglycerides, poloxamers, polysorbates, stearic acid, sodium lauryl sulfate, triacetin, triethyl citrate, lecithins, mineral oil, talc, kaolin, and combinations thereof.

An ordinarily skilled artisan will recognize that additional ingredients; e.g., preservatives, lubricants, colorants, flavors, emulsifiers, buffering agents, antioxidants, etc.; may also be included in an enteric coating composition as described herein. Examples of preservatives include, but are not limited to, benzalkonium chloride, benzoic acid, benzyl alcohol, benzoates, sorbates, nisin, natamycin, calcium priopionate, sorbic acid, sodium benzoate, methyl paraben, ethyl paraben, propyl paraben, butyl paraben, phenol, cresol, quaternary ammonium salts, potassium sorbate, and combinations thereof. Examples of lubricants include, but are not limited to, talc, metallic stearates, silicon dioxide, sodium stearyl fumarate, palmitic acid, fatty acid esters, fatty acids, fatty alcohols, mineral oil, paraffins, leucine, polyethylene glycols, metallic lauryl sulfates, stearic acid, hydrogenated vegetable oil, and combinations thereof.

Examples of colorants include dyes, lakes, and pigments and may include, but are not limited to, titanium dioxide, iron oxides, dyes such as, for example, FD&C Lakes, Carmine Lake, FD&C Blue no. 1, FD&C Red no. 3, FD&C Red no. 40, FD&C Yellow no. 5, FD&C Yellow no. 6, FD&C Green no. 3, alumina, talc, annatto extract, calcium carbonate, canthaxanthin, caramel, β-carotene, carmine, dihydroxyacetone, tumeric oleoresin, cochineal extract, gardenia yellow, gardenia blue, beet powder, grape skin extract, riboflavin, chlorophyll-containing extracts, pearlescent pigments, SensiPearl™ Blue, Silver, and Bright Silver (available from Sensient Colors, Inc.), natural colorants, and the like. Other examples of colorants are found in 21 C.F.R. §§73 and 74, which are hereby fully incorporated by reference.

Examples of flavors may be synthetic or artificial flavors, natural flavors or any mixture thereof and may include, but are not limited to, flavonoids, antioxidants, natural flavorants, synthetic flavorants, bioflavenoids, flavones, flavone, flavonol, flavanonol, isoflavones, ethyl vanillin, tangerine flavor, lemon flavor, lemon extract, liquid caramel, spearmint oil, orange flavor, almond, amaretto, apple, green apple, apple-cherry-berry, apple-honey, apricot, bacon, balls of fire, banana, barbeque, beef, roast beef, beef steak, berry, berry blue, birch beer/spruce beer, blackberry, bloody mary, blueberry, boysenberry, brandy, bubble gum, butter, butter pecan, buttermilk, butterscotch, candy corn, cantaloupe, cantaloupe lime, caramel, carrot, cassia, caviar, celery, cereal, champagne, cherry, cherry cola, cherry maraschino, wild cherry, black cherry, red cherry, cherry-cola, chicken, chocolate, chocolate almond, cinnamon spice, citrus, citrus blend, citrus-strawberry, clam, cocoa, coconut, toasted coconut, coffee, coffee almond, cola, cola-vanilla, cookies & cream, cool, cotton candy, cranberry, cranberry-raspberry, cream, cream soda, dairy type cream, creme de menthe, cucumber, black currant, dulce de leche, egg nog, pork fat, type fat, anchovy fish, herring fish, sardine fish, frankfurter, fiery hot, fried garlic, sauteed garlic, gin, ginger ale, ginger beer, graham cracker type, grape, grape grapefruit, grapefruit-lemon, grapefruit-lime, grenadine, grill, guarana, guava, hazelnut, honey, hot, roasted honey, ice cream cone, jalapeno, key lime, kiwi, kiwi-banana, kiwi-lemon-lime, kiwi-strawberry, kola champagne, lard type, lemon, lemon custard, lemonade, pink lemonade, lemon-lime, lime, malt, malted milk, mango, mango-pineapple, maple, margarita, marshmallow, meat type, condensed milk, cooked milk, mint, mirepoix, mocha, mochacinna, molasses, mushroom, sauteed mushroom, muskmelon, nectarine, neopolitan, green onion, sauteed onion, orange, orange cordial, orange creamsicle, orange creme, orange peach mango, orange strawberry banana, creamy orange, mandarin orange, orange-passion-guava, orange-pineapple, papaya, passion fruit, peach, peach mango, peanut, roasted peanut, pear, pecan danish type, pecan praline, pepper, peppermint, pimento, pina colada, pina colada/pineapple-coconut, pineapple, pineapple-orange, pistachio, pizza, pomegranate, pork fat type, baked potato, prune, punch, citrus punch, tropical punch, cherry fruit punch, grape punch, raspberry, black raspberry, blue raspberry, red raspberry, raspberry-blackberry, raspberry-ginger ale, raspberry-lime, roast type, root beer, rum, sangria, sarsaparilla, sassafras, sausage, sausage pizza, savory, seafood, shrimp, hickory smoke, mesquite smoke, sour, sour cream, sour cream and onion, spearmint, spicy, strawberry, strawberry margarita, jam type strawberry, strawberry-kiwi, burnt sugar, sweet, supersweet, sweet & sour, tallow, tamarind, tangerine-lime, tangerine, tea, tequila type, toffee, triple sec, tropical fruit mix, turkey, tutti frutti, vanilla, vanilla cream, vanilla custard, french vanilla, vegetable, vermouth, vinegar, balsamic vinegar, watermelon, whiskey, wildberry, wine, and yogurt, and the like. Other examples of flavors are found in 21 C.F.R. §§172.510, 172.515, 172.520, 172.530, 172.535, 172.575, 172.580 and 172.585, which are hereby fully incorporated by reference. A variety of food grade flavors are commercially available from Sensient Flavors Inc. in Indianapolis, Ind., Givaudan SA in Cincinnati, Ohio, and International Flavors & Fragrance in New York, N.Y.

Examples of sweeteners and/or taste maskants may include, but are not limited to, smoothenol, rosemary extract, aspartame, sucrose, honey, Magnasweet™, saccharin, sucralose, and the like. Examples of emulsifiers include, but are not limited to, polysorbates (polyethoxylated sorbitan fatty acid derivatives) such as, for example, polysorbate 80; polyglyceryl 10 laurate; mono- and di-glycerides; propylene glycol; sodium lauryl sulfate; additives of propyl gallate and citric acid and stabilizers therein; alcohol; and combinations thereof. Examples of buffering agents include, but are not limited to, sodium citrate. Examples of antioxidants include, but are not limited to, tocopherol, rosemary extract, and combinations thereof. Examples of solvents include, but are not limited to, ethanol, water, and combinations thereof.

The enteric coating composition may form an aqueous solution having a pH of at least about 5, at least about 6, and at least about 7. The enteric coating composition may form an aqueous solution having a pH of less than about 9, less than about 8, and less than about 7. This includes, for example, about 5 to about 9, about 6 to about 8, and about 7 to about 8. The pH of the enteric coating composition, or of components of the enteric coating composition, may be adjusted and selected to maintain a useable solution or suspension. Alternative, the enteric coating composition may be in the form of a suspension, which may have a pH of less than 4, less than 3, and a pH of at least about 2, at least about 3, and at least about 4. When applied to a core solid dosage form comprising an active agent, the enteric coating composition is preferably solid and more preferably stable. Stable as used herein refers to the ability of the solid enteric coating composition to prevent the release of about 90% of the active agent at a pH less than 2 for at least 60 minutes.

Active Agents

The enteric coating composition may be applied to core solid dosage forms containing one or more active agents, such as a pharmaceutical, nutraceutical, dietary supplement, nutritional supplement, medical food, fruit, vegetable, agricultural product, or industrial product, so as to form an enteric coating around the core dosage form.

Non-limiting application techniques are described herein. In some embodiments, a coating pan may be charged with core capsules, tablets, mini-tablets and/or softgels. The bed may be warmed to at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., and at least about 30° C. The bed may be warmed to less than about 42° C., less than about 41° C., less than about 40° C., less than about 39° C., less than about 38° C., less than about 37° C., less than about 36° C., less than about 35° C., less than about 34° C., less than about 33° C., less than about 32° C., less than about 31° C., less than about 30° C., and less than about 25° C. This includes, for example, about 18° C. to about 42° C., about 20° C. to about 40° C., and about 25° C. to about 37° C. The processing parameters may be as set forth below.

The inlet temperature may be at least about 35° C., at least about 40° C., at least about 41° C., at least about 42° C., at least about 43° C., at least about 44° C., at least about 45° C., at least about 50° C., and at least about 55° C. The inlet temperature may be less than about 65° C., less than about 60° C., less than about 59° C., less than about 58° C., less than about 57° C., less than about 56° C., less than about 55° C., less than about 54° C., less than about 53° C., less than about 52° C., less than about 51° C., and less than about 50° C. This includes inlet temperatures, for example, from about 35° C. to about 65° C., about 40° C. to about 65° C., and about 45° C. to about 60° C.

The outlet temperature may be at least about 20° C., at least about 25° C., at least about 28° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., and at least about 42° C. The outlet temperature may be less than about 50° C., less than about 45° C., less than about 44° C., less than about 43° C., less than about 42° C., less than about 41° C., and less than about 40° C. This includes, for example, outlet temperatures from about 20° C. to about 50° C., from about 25° C. to about 45° C., from about 28° C. to about 45° C., from about 30 to about 45° C., or from about 35° C. to about 42° C.

The atomization pressure may be at least about 10, at least about 15, at least about 20, at least about 25, and at least about 30 psi. The atomization pressure may be less than about 45, less than about 40, less than about 35, and less than about 30 psi. This includes, for example, atomization pressures from about 10 psi to about 45 psi, from about 15 psi to about 40 psi, and from about 30 psi to about 40 psi. The air volume may be from about 50 cfm to about 500 cfm.

The pan speed may be from about 3 rpm to about 22 rpm, about 8 rpm to about 20 rpm, about 11 rpm to about 19 rpm, and about 12 rpm to about 18 rpm. The spray rate may be from about 0.5 g/min/kg to about 10 g/min/kg, and about 2 g/min/kg to about 50 g/min/kg.

The solution solids may at least about 5%, at least about 6%, at least about 7%, at least about 8%, at least about 9%, at least about 10%, at least about 11%, at least about 12%, at least about 13%, at least about 14%, at least about 15%, at least about 20%, and at least about 25%. The solution solids may be less than about 35%, less than about 30%, less than about 20%, and less than about 15%.

The coating time may be from about 5 minutes to about 3 hours, about 10 minutes to about 2.5 hours, about 0.5 hours to about 2 hours, and about 1 hour to about 1.5 hours.

The enteric coating composition may be applied to a pharmaceutical or nutraceutical agent by loading at least about 5 Kg, at least about 10 Kg, at least about 11 Kg, at least about 12 Kg, at least about 13 Kg, at least about 14 Kg, or at least about 15 Kg of the pharmaceutical or nutraceutical agent into a vented coating pan, such as, for example, a 24″ side-vented coating pan (Compu-Lab 24). In some embodiments, the side-vented coating pan can be a 48″ pan, or a 60″ pan and the loading increase accordingly. The enteric coating composition can be applied under the following conditions. The inlet temperature may be about 40° C. to about 65° C., the outlet temperature may be from about 20° C. to about 50° C., from about 25° C. to about 45° C., from about 30° C. to about 45° C., or from about 35° C. to about 42° C., the pan charge may be at least about 5 Kg, at least about 10 Kg, and at least about 15 Kg, the atomization pressure may be about 15 psi to about 40 psi, the air volume may be about 50 cfm to about 500 cfm, the pan speed may be about 3 rpm to about 11 rpm, the spray rate may be about 30 g/min to about 100 g/min, the solution solids may be about 10% to about 15%, the weight gain may be about 2.5% to about 4.5%, the coating efficiency may be about 75% to about 99%, and the coating time may be about 60 minutes to about 180 minutes. The enteric coating composition (in solution or suspension form) may, at a suitable concentration which is spraying system dependent, be sprayed using commercially available equipment to form films on dosage units.

The enteric coating composition may be in the form of a spray solution or a suspension, and may be applied to core dosage form containing the pharmaceutical or nutraceutical agent using a vented coating pan (i.e., sprayed). The enteric coating composition may be applied to coat core substrates at a variety of coating temperatures (e.g., from about 28° C. to about 60° C.) and spray rates. The film may not be tacky and spray rate and temperature may not be factors. The solution can be allowed to dry, forming a dry enteric coating that can protect the pharmaceutical or nutraceutical agent from being attacked by acid of a stomach.

The enteric coating compositions may be used in food, pharmaceutical or nutraceutical applications intended for use in mammals, including, without limitation, rodents, canines, felines, non-human primates, ungulates, and humans. They may coat pharmaceutical or non-pharmaceutical dosage units. The enteric coating compositions may form an enteric coating that resists acid but disintegrates in neutral or mildly alkaline conditions. It may possess the properties of an enteric film, and have a controlled release profile such that it will release in an environment having a specified pH. The enteric coating may produce a controlled release profile in an environment having a selected pH based on a resin:polymer ratio. The enteric coating composition may be in the form of a spray solution or a suspension, and may be applied to a pharmaceutical or nutraceutical agent using a vented coating pan (i.e., sprayed). The enteric coating composition may be applied to coat substrates at a variety of coating temperatures (e.g., from about 28° C. to about 60° C.) and spray rates. The film may not be tacky and spray rate and temperature may not be factors. The solution can be allowed to dry, forming a dry enteric coating that can protect the active agent, e.g., pharmaceutical agent, nutraceutical agent, etc., from being released into an acidic environment, e.g., the stomach. Preferably, the enteric coating composition dissolves and releases the active agent at a pH of about 5.5 to about 6.5.

### EXAMPLES

### Example 1

### Preparation of Coating Dispersion and Coating Parameters

An organic acid salt (potassium sorbate or sodium benzoate) was in water and the resulting solution was gradually added to a shellac solution (Marcoat 125) while stirring.

### Example 2

### Enteric Coat Compositions

### Example 2.1

### Coating Solution for a pH 5.5 Coating System

| | | | | | % | |
|---|---|---|---|---|---|---|
| | Amount | % w/w | Solids | Solid/ | polymer: | % w/w |
| Ingredient | (g) | composition | (%) | batch | acid | solution |
| Marcoat | 540 | 36 | 25 | 135 | 60 | 9 |
| 125 | | | | | | |
| Sodium | 90 | 6 | 100 | 90 | 40 | 6 |
| benzoate | | | | | | |
| Deionized | 870 | 58 | 0 | 0 | — | 0 |
| water | | | | | | |
| Total | 1500 | 100 | | 225 | 100 | 15 |

### Example 2.2

### Coating Solution for a pH 5.5 Coating System

| | Amt/ | | | | % | % solid |
|---|---|---|---|---|---|---|
| | batch | % w/w | Solids | | solids | in |
| Ingredient | (g) | composition | (%) | solid (g) | w/w | solution |
| Marcoat | 540.0 | 15.4 | 25 | 135.0 | 38.46 | 3.86 |
| 125 | | | | | | |
| Plasacryl | 67.5 | 1.9 | 20 | 13.5 | 3.85 | 0.39 |
| Potassium | 202.5 | 5.8 | 100 | 202.5 | 57.69 | 5.79 |
| sorbate | | | | | | |
| Deionized | 2,690.0 | 76.9 | 0 | 0 | — | — |
| water | | | | | | |
| Total | 3,500.0 | 100.0 | — | 351.0 | 100.00 | 10.04 |

### Example 2.3

### Coating Solution for a pH 6.5 Coating System

| | Amt/batch | % w/w |
|---|---|---|
| Ingredient | (g) | composition |
| Marcoat 125 | 540 | 15.43 |
| Potassium sorbate | 90 | 2.57 |
| Deionized water | 2,870 | 82.00 |
| Total | 3,500 | 100.00 |

### Example 2.4

### Coating Solution for a pH 6.5 Coating System

| | Amt/batch | % w/w |
|---|---|---|
| Ingredient | (g) | composition |
| Marcoat 125 | 540 | 15.43 |
| Sodium benzoate | 34 | 0.97 |
| Deionized water | 2,926 | 83.60 |
| Total | 3,500.00 | 100.00 |

### Example 2.5

### Coating Solution for a pH 5.5 Coating System

| | Amt/batch | % w/w |
|---|---|---|
| Ingredient | (g) | composition |
| Orange dewaxed | 135.0 | 3.86 |
| Shellac | | |
| Sodium bicarbonate | 30.0 | 0.86 |
| Potassium sorbate | 202.5 | 5.79 |
| DI water | 3,132.5 | 89.50 |
| Total | 3,500.00 | 100.00 |

### Example 3

### Coating of Core Tablets

Dispersions containing 20% acid were able to be sprayed as a 20% total solids dispersion without gelling. For higher acid proportions the concentration of solid in the coating dispersion was reduced to 10-15% (depending on the proportion of the acid) to allow spraying without gelling.

Five to ten kilograms of 500 mg core tablets comprising the active agent Metformin were charged into a coating pan Manesty Acela Cota 24 inch pan). The inlet temperature was set to between 50-65° C. (preferred range 50-55° C.), the pressure difference adjusted to 1.2 bar and the core tablet heated to a bed temperature of 30-35° C. while the pan was rotated at 14-16 RPM. The dispersion was continuously stirred during the spray coating step.

Alternatively, the shellac dispersion and the acid solution were prepared separately and co-sprayed using one spray gun where the two solutions were pumped through a T- or Y-connector allowing the two dispersions to mix just before the atomization.

A subcoat/seal coat (e.g. 7.5% Hypromellose E6 solution or Opadry Clear) was applied to the core tablets to about 2-3% target weight gain. The need for sub-coat is established during development of the product.

After the seal coat, an enteric coating solution was sprayed at a rate of 2-5 g/min/kg onto the tablet bed. The spray rate, inlet temperature and/or pressure difference were adjusted to minimize over-wetting and/or sticking of the core tablets during coating and to keep the outlet/product bed temperature between 20-30° C. Spraying was continued until 2.5% (range 2.2 to 3%) target weight gain was attained.

A top-coat consisting of GRAS film forming ingredients such as hydroxypropylmethyl-cellulose (HMPC), polyvinyl alcohol (PVA), Shellac or ethylcellulose with sodium alginate or GRAS-listed Eudragit E was applied to reduce tackiness on storage at accelerated conditions, (especially for enteric coating compositions with high sorbate or benzoate content) and/or to increase acid-resistance of the tablets during in vitro dissolution or residence in the stomach upon administration to a patient. After application of the top-coat, the coated tablets were cured at a bed temperature of 35-40° C. for about 10 minutes, allowed to cool to ambient temperature and collected into suitable containers.

### Example 4

### Testing and Results

Metformin solid dosage forms coated with enteric coating compositions comprising 10% (wt/wt) to 40% (wt/wt) potassium sorbate at a target weight gain of 2% to 4% were tested for stability in an acid test and for release of metformin at various pHs. The data show that metformin tablets coated to 2.2, 2.4, 2.6 and 3% target weight gains were stable at a pH of 1.2 for at least 2 hours, but released metformin at a pH between 5.5 and 6.5. Shown in Table 1 is the percentage of metformin released after 1 or 2 hours at a pH of less than 2 from metformin dosage forms coated with an enteric coating composition comprising shellac and 20% (wt/wt) or 10% (wt/wt) potassium sorbate at a target weight gain of 2, 3, or 4 or 6.

TABLE 1
MPS 20% Coated tablets in acid test (less than 10% release @ 2 hours)

| | Formu- | Formu- | For- | For- | For- | For- |
|---|---|---|---|---|---|---|
| | lation | lation | mulation | mulation | mulation | mulation |
| Hrs | 20-2% | 20-3% | 20-4% | 10-2% | 10-4% | 10-6% |
| 1 Hr | 15% | 2.5% | 0.4% | 15% | 1.0% | 0.8% |
| 2 Hr | 34% | 9.3% | 0.4% | 53% | 7.6% | 8.3% |

Shown in Table 2 is the percentage of metformin released after 1 or 2 hours at a pH of less than 2 from metformin dosage forms coated with an enteric coating composition comprising shellac and 40% (wt/wt) potassium sorbate at a target weight gain of 2.2, 2.4, 2.6, 2.8 or 3.

TABLE 2
MPS 40% Coated tablets in acid test
(less than 10% release @ 2 hours).

| | | Form- | | Form- | |
|---|---|---|---|---|---|
| | Formulation | ulation | Formulation | ulation | Formulation |
| Hrs | 40-2.2% | 40-2.4% | 40-2.6% | 40-2.8% | 40-3.0% |
| 1 Hr | 9.8% | 6.8% | 6.2% | 4.4% | 1.2% |
| 2 Hr | 12% | 11% | 10% | 5.6% | 4.4% |

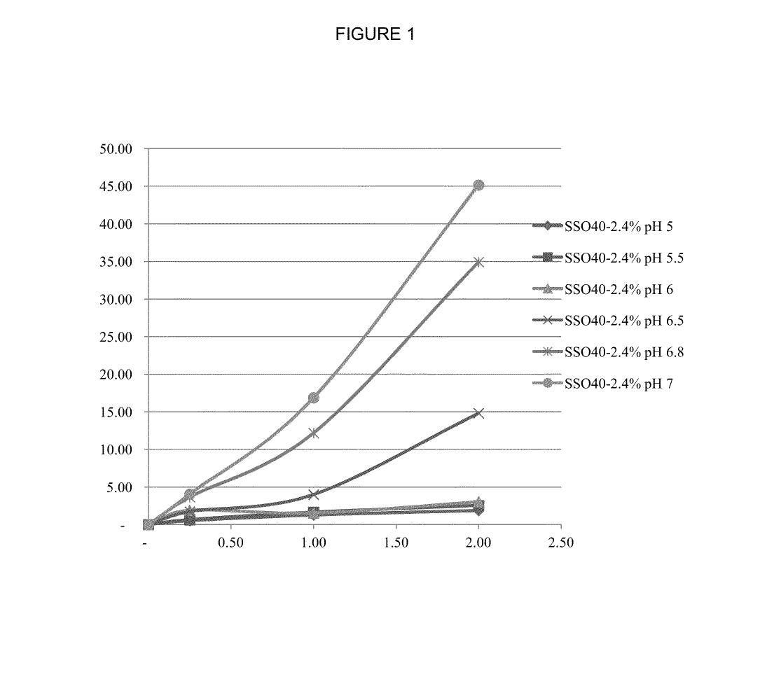

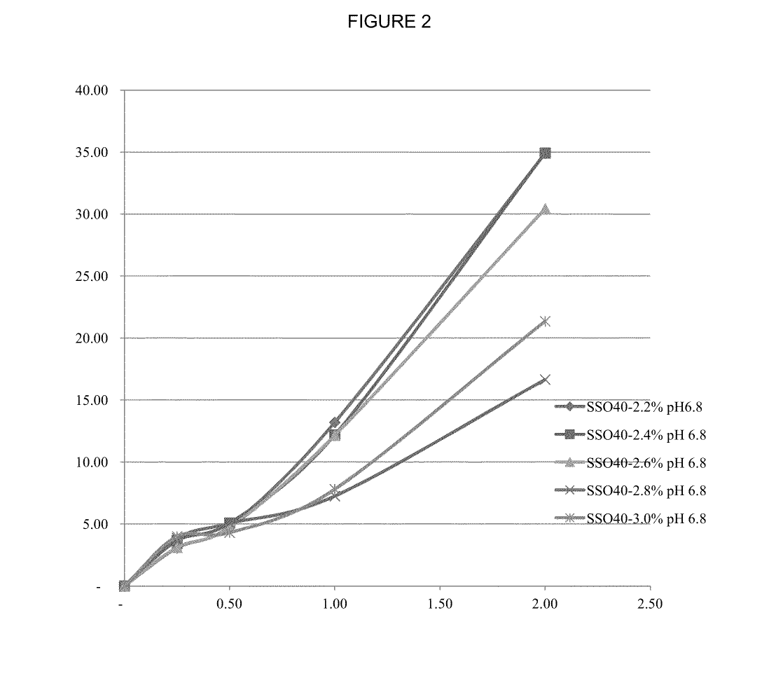

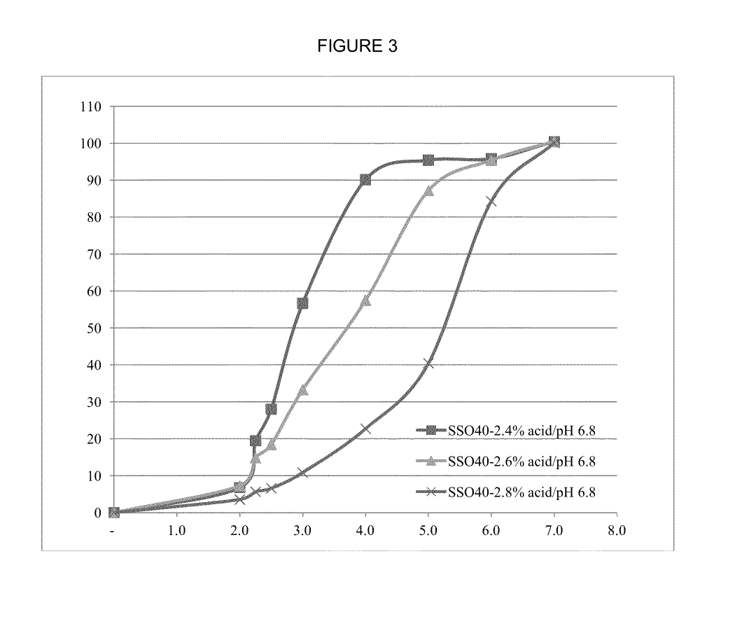

FIGS. 1-3 provide the release profiles of metformin tablets coated with various enteric coating composition comprising shellac 60%:sorbate 40% at a pH of 6.5 or greater with or without an acid pre-incubation step.

The data show that tablets coated with an enteric coating comprising shellac 60%:sorbate 40% at 2% target weight gain were not stable, but tablets coated at 2.2, 2.4, 2.6 and 3% target weight gain were acid stable. Additionally, such tablets were able to release metformin at a pH of 6.5 and above.

Metformin dosage forms coated with enteric coating compositions comprising shellac 60%: benzoate 40% (wt/wt) at a target weight gain of 2.0 to 2.9% were tested for stability in an acid test and for release of metformin at a pH of 1.2. Shown in Table 3 is the percentage of metformin released after 1 or 2 hours at a pH of 1.2 from the metformin dosage forms.

TABLE 3
MBS 40% Coated tablets in acid test

| | Formu- | Formu- | For- | For- | For- | For- |
|---|---|---|---|---|---|---|
| | lation | lation | mulation | mulation | mulation | mulation |
| | SB40- | SB40- | SB40- | SB40- | SB40- | SB40- |
| Hrs | 2.0% | 2.2% | 2.4% | 2.6% | 2.8% | 2.9% |
| 1 | 2.23% | 2.51% | 2.67% | 2.42% | 2.74% | 2.89% |
| 2 | 2.41% | 2.72% | 2.81% | 2.58% | 2.85% | 3.16% |

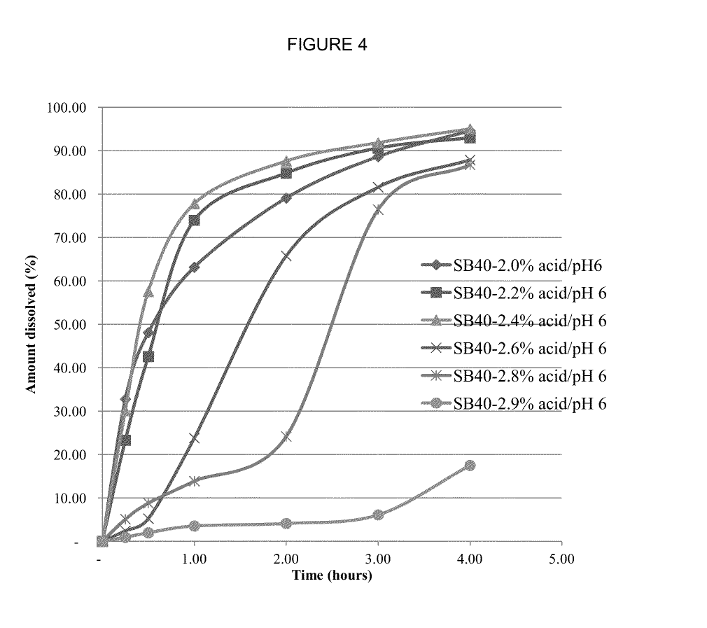

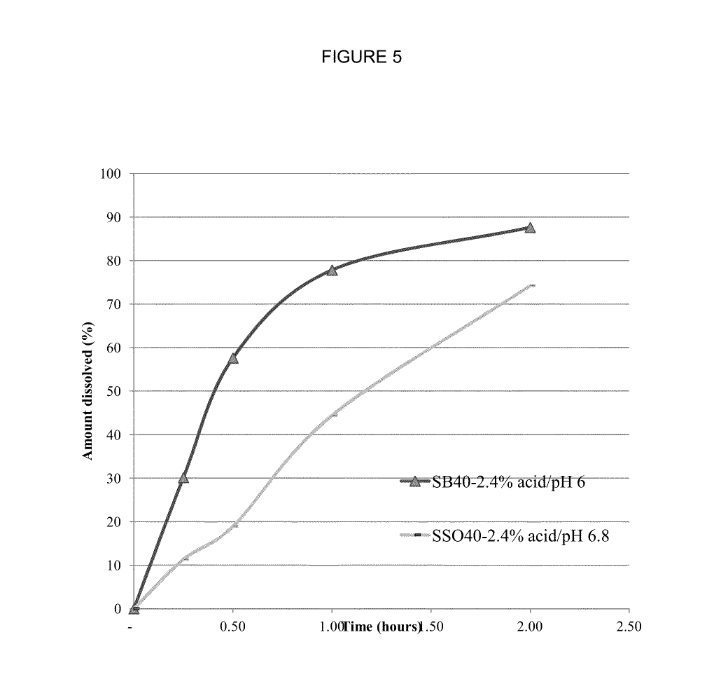

The results shown in Table 3 and FIGS. 4 and 5 demonstrates that Metformin tablets coated with shellac 60%: sodium benzoate 40% resists drug release in 0.1M HCl for at least 2 hours but released the drug in buffer at pH 6 (Table 3 and FIG. 4). Furthermore, the data in FIG. 5 shows that benzoate-based coating systems release the drug faster and at a lower pH compared with sorbate-based systems in line with their pKa values.

### Example 5

### Release of Drugs from Mini-Tablets Coated with Shellac-Sorbate System

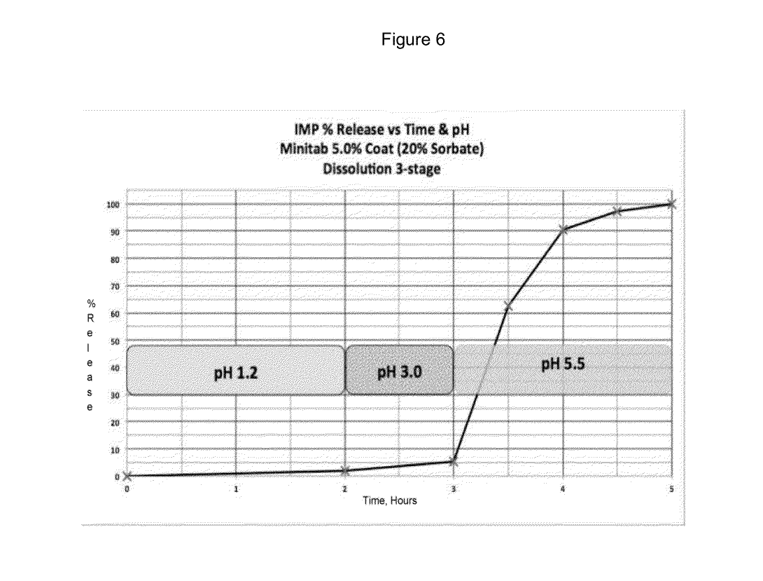

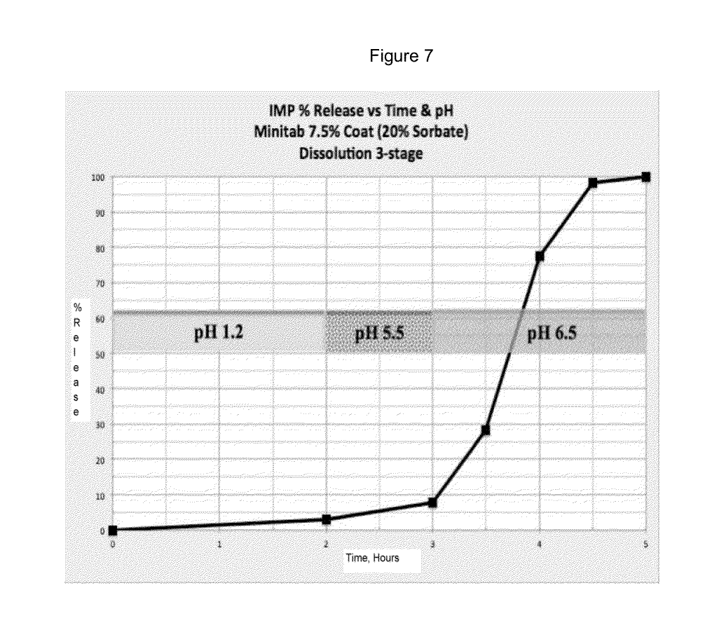

Mini-tablets (40 mg, 3 mm diameter, 4 mm thickness) containing 6.0 mg disodium inosinate (IMP), as a model nutritional compound, are prepared and coated with shellac-sorbate coating system. The results show that the pH at which the drug is released depends on both the ratio of the shellac to sorbate and the amount of coating deposited on the mini-tablets. Without being limited by any particular mechanism, higher proportion of sorbate in the coating system lowers the pH at which the drug is released. In addition, for the same shellac:sorbate ratio, mini-tablets coated to higher coating levels released the drug at higher pH. See, FIGS. 6 and 7.

As provided in the above example, tablets are either coated without a sub-coat or with a sub-coat which does not significantly impact the active agent release from the core tablet. Release-modifying materials such as EUDRAGIT® E, EUDRAGIT® NM or similar polymers can be used as sub-coat materials to slow down the release of the active agent from the core tablet in order to obtain precise targeting of the various regions of the gut.

All patents and patent publications referred to herein are hereby incorporated by reference.

Certain modifications and improvements will occur to those skilled in the art upon a reading of the foregoing description. It should be understood that all such modifications and improvements have been deleted herein for the sake of conciseness and readability but are properly within the scope of the following claims.

1. An enteric coating composition comprising about 20% to about 90% (w/w %) of film former and about 10% to 80% (wt/wt %) of pore former, wherein said pore former comprises at least one organic acid having a pKa greater than 3.0, and wherein said composition may be coated onto a core solid dosage form containing an active agent to provide an enteric coating thereon for release or dissolution at a pH between 4.5 and 7.

2. The coating composition according to claim 1, wherein said film former is selected from the group consisting of a pH-dependent polymer, a pH-independent polymer, and a water-insoluble polymer.

3. The coating composition according to claim 1, wherein said film former is Generally Recognized as Safe (GRAS).

4. The coating composition according to claim 3, wherein said film former is selected from the group consisting of shellac and ethylcellulose.

5. The coating composition of claim 1, wherein said organic acid is Generally Recognized as Safe (GRAS).

6. The coating composition of claim 5, wherein the organic acid is selected from the group consisting of sorbic acid, benzoic acid and succinic acid, or a salt thereof.

7. The coating composition of claim 1, further comprising about 0.01% to about 10.0% (wt/wt %) plasticizer.

8. The coating composition of claim 1, further comprising about 0.01% to about 10.0% (wt/wt %) anti-caking agent.

9. The coating composition of claim 1, wherein the composition is in the form of a spray solution or a suspension.

10. The coating composition of claim 9, wherein the composition is a spray solution.

11. The coating composition of claim 10, wherein the composition has a pH of between 7 and 8.

12. The coating composition of claim 9, wherein the composition is a suspension.

13. The coating composition of claim 12, wherein the composition has a pH between 2 and 4.

14. The coating composition as in any one of claims 1-8, wherein the composition is in the form of a solid enteric coating.

15. The coating composition of claim 14, wherein said solid enteric coating is stable.

16. A method of preparing a delayed-release solid dosage form, comprising applying the enteric coating composition according to claim 1 to a core solid dosage form comprising an active agent.

17. A delayed-release solid dosage form comprising the enteric coating according to claim 1 applied to a core solid dosage form containing an active agent.

18. The delayed-release solid dosage form of claim 16, wherein the active agent is a nutraceutical.

19. The delayed-release solid dosage form of claim 16, wherein said core solid dosage form is a capsule, tablet, mini-tablet, soft gel or granules for suspension.

20. A method for delivering an active agent to the intestine of a subject in need thereof, comprising administering to the subject a delayed-release solid dosage form comprising a core solid dosage form containing at least one active agent, wherein said core solid dosage form is enveloped by an enteric coating comprising at least one film former and at least one pore former, wherein said pore former comprises an organic acid having a pKa greater than about 3.0.
